(19) United States
(12) Patent Application Publication
(10) Pub. No.: US 2021/0360933 A1
(43) Pub. Date: Nov. 25, 2021

(54) Microbe-Based Products for Controlling Fusarium Infections in Plants and Agricultural Products

(71) Applicant: Locus Agriculture IP Company, LLC, Solon, OH (US)

(72) Inventors: Sean FARMER, North Miami Beach, FL (US); Ken ALIBEK, Solon, OH (US); Paul S. ZORNER, Encinitas, CA (US); Alibek MOLDAKOZHAYEV, Solon, OH (US); Samal IBRAGIMOVA, Solon, OH (US)

(21) Appl. No.: 16/975,118

(22) PCT Filed: Feb. 26, 2019
(86) PCT No.: PCT/US2019/019595

§ 371 Date: Aug. 22, 2020

Related U.S. Application Data
(60) Provisional application No. 62/636,273, filed on Feb. 28, 2018.

(51) Int. Cl. A01N 63/32; A01P 3/00; A01N 25/30

(52) U.S. Cl. CPC A01N 63/32; A01N 25/30; A01P 3/00

(57) ABSTRACT

Compositions and methods are provided for controlling Fusarium fungal infections in plants. In particular, the subject invention relates to treatments for plant pathogenic fungal infections using microbes and/or their growth by-products. In a specific embodiment, the subject invention can be used to treat and/or prevent infection from a strain of Fusarium oxysporum that causes Fusarium Wilt and/or Panama Disease in Musa plants, although control of other Fusarium infections in other plants and/or agricultural products is also envisioned.

### CROSS-REFERENCE TO RELATED APPLICATION

This application claims priority to U.S. Provisional Patent Application No. 62/636,273 filed Feb. 28, 2018, which is incorporated herein by reference in its entirety

### BACKGROUND OF THE INVENTION

Bananas and plantains are perennial giant herbs belonging to the genus Musa. More than 100 billion bananas are consumed by humans each year, making them one of the most popular agricultural products worldwide. Approximately 10% of the world's production of bananas enters the export market to generate an important source of income for tropical and subtropical regions. As with many agricultural crops around the world, the vast majority of internationally traded bananas are a monoculture. Most commercial banana plants were collected from nature, domesticated and maintained by clonal propagation, which led to plantations comprised of plants that are nearly genetically identical to one another.

One cultivar, the Cavendish, currently makes up the majority of exported bananas worldwide. Unlike other agricultural monocultures, however, the Cavendish did not gain worldwide prominence because of its superior taste, hardiness, or shelf-life. Instead, the Cavendish became globally dominant because of its resistance to a fungal infestation, commonly known as Panama disease, which devastated the banana industry in the 1950s and drove the previous monoculture to commercial extinction by 1965. Now, decades later, a new strain of the same fungal infestation-Fusarium oxysporum f sp. cubense—is plaguing Cavendish crops, and rapidly spreading worldwide.

Fusarium is a soil pathogen that is propagated by asexual spores. It infests the root system of plants and is drawn up into a plant through its vascular system. The fungus develops further colonies within the xylem of banana plants, thus blocking the internal flow of nutrients and water. This blockage leads to wilt (i.e., “Fusarium wilt”) and eventual death.

The strain of Fusarium that causes Fusarium wilt in Cavendish bananas, along with the previous iteration that caused Panama Disease, is immune to all known fungicides and, because it is soil-borne, currently leaves banana cultivators with little option but to burn down infested fields and plant some other crop. Given the potentially dire consequences of the return of Fusarium infestations, and the failures of previous methods of treatment or control (including shifting cultivation to an apparently resistant cultivar), new approaches need to be explored for the survival of the banana industry.

To address the global needs for sustainable methods of producing food and consumable products, microbes such as bacteria, yeast and fungi, as well as their byproducts, are becoming increasingly useful in many settings, including agriculture and horticulture, animal husbandry, forestry, and remediation of soils, waters and other natural resources. For example, farmers are embracing the use of biological agents such as live microbes, bio-products derived from these microbes, and combinations thereof, as biopesticides and biofertilizers.

There is a continuing need for improved, non-toxic and environmentally-friendly methods of enhancing and protecting crop production at a low cost. In particular, there is a need for products to combat Fusarium in, for example, banana plantations, without compromising the environment in which they are used or the health of the people who are exposed to the products.

### BRIEF SUMMARY OF THE INVENTION

The subject invention provides microbes, as well as by-products of their growth, such as biosurfactants. The subject invention also provides methods of using these microbes and their by-products, as well as methods and systems for producing them. Advantageously, the microbe-based products and methods of the subject invention are environmentally-friendly, non-toxic and cost-effective.

In one embodiment, the subject invention provides fungicidal compositions and/or inocula comprising a non-pathogenic microorganism and/or a growth by-product thereof. Also provided are methods of cultivating the microorganism and/or growth by-products of the fungicidal composition.

In preferred embodiments, the subject invention provides fungicidal compositions and methods of their use. Specifically, the subject invention provides for treatment and/or prevention of infection by Fusarium pathogens such as, e.g., Fusarium oxysporum, that infect, e.g., banana crops.

In one embodiment, a fungicidal composition is provided, comprising one or more yeast strains and/or growth by-products thereof. In preferred embodiments, the one or more yeast strains are selected from Pichia clade yeasts. Even more preferably, the yeast strains are selected from Pichia occidentalis and Pichia anomala (a.k.a. Wickerhamomyces anomalus).

The microorganisms of the subject fungicidal compositions can be obtained through cultivation processes ranging from small to large scale. These cultivation processes include, but are not limited to, submerged cultivation/fermentation, solid state fermentation (SSF), and combinations thereof.

The fungicidal composition can comprise fermentation medium and/or purified or unpurified growth by-products, such as biosurfactants, killer toxins, enzymes and/or other metabolites. The microbes can be live or inactive.

The composition is preferably formulated for application to soil, seeds, whole plants or plant parts (including, but not limited to, roots, tubers, stems, flowers, leaves and the vascular system). In certain embodiments, the composition is formulated as, for example, concentrated liquid, dust, granules, microgranules, pellets, wettable powder, flowable powder, emulsions, microcapsules, oils, or aerosols. In preferred embodiments, the composition is formulated to be suitable as a soil treatment.

To improve or stabilize the effects of the composition, it can be blended with suitable adjuvants and then used as such, or after dilution, if necessary. In preferred embodiments, the composition is formulated as a liquid, or as dry powder or granules that can be mixed with water and other components to form a liquid product. In one embodiment, the composition can further comprise a source of protein and other nutrients, such as, for example, carbon, nitrogen, vitamins, micronutrients and amino acids, for enhanced growth of the beneficial microorganisms and production of fungicidal growth by-products.

The composition can be used either alone or in combination with other compounds for efficiently enhancing the antifungal effects. For example, in some embodiments, the composition can comprise additional components, such as commercial and/or homemade herbicides, fertilizers, pesticides, repellants and/or soil amendments that are compatible with the one or more fungicidal microorganisms, and/or microbial growth by-products. In one embodiment, the composition can further comprise, and/or be used alongside, a biosurfactant composition.

In one embodiment, the composition can be mixed and/or applied with a commercial fertilizer, such as Scott's Miracle-Grog, or another source of plant nutrients (e.g., nitrogen-phosphorous-potassium (NPK) and micronutrients). The exact materials and the quantities thereof can be determined by an agricultural and/or microbiological scientist having the benefit of the subject disclosure.

In one embodiment, methods are provided for controlling Fusarium fungi in a plant and/or its surrounding environment, wherein one or more non-pathogenic microorganisms, and/or their growth by-products, are contacted with the plant and/or its surrounding environment.

The method can comprise contacting a fungicidal composition of the subject invention, comprising one or more microorganisms and/or a growth by-product thereof, with the plant and/or its surrounding environment. Preferably, the one or more microorganisms and/or their growth by-products are capable of fungicidal action. In one embodiment, the one or more fungicidal microorganisms are selected from Pichia yeasts, for example, P. occidentalis and P. anomala (a.k.a. Wickerhamomyces anomalus), as well as mutants thereof.

Advantageously, the subject method can even be used to enhance health, growth and/or yields in plants having compromised immune health due to an infection from a Fusarium fungus or from an environmental stressor. Furthermore, the subject method can be used to reduce the amount of plant and/or crop loss due to plant damage and/or death caused by Fusarium infections. Even further, the subject methods can be used for controlling fungal infections in or on plant tissue without phytotoxicity.

In preferred embodiments, the subject method can be used for controlling Fusarium fungi. In one embodiment, the subject method can be used to control Fusarium oxysporum, and/or Fusarium oxysporum spp. cubense. Thus, the method can be used for treating and/or preventing, for example, Panama Disease and/or Fusarium wilt in banana plants. In additional embodiments, the method can be used for controlling Fusarium fungi, such as, for example, F. avenaceum, F. bubigeum, F. culmorum, F. graminearum, F. langsethiae, F. oxysporum, F. proliferatum, F. sporotrichioides, F. poae, F. reseum, F. solani, F. tricinctum, F. verticillioides, F. virguliforme, F. xylariodides and any other Fusarium fungi that can infect plants.

The methods can comprise applying nutrients to enhance the growth of the one or more fungicidal microorganisms and/or production of fungicidal growth by-products. Such nutrients can include, for example, sources of carbon, nitrogen, potassium, phosphorus, magnesium, proteins, micronutrients, vitamins and/or amino acids.

In certain embodiments, the fungicidal composition is contacted with a plant part. In a specific embodiment, the composition is contacted with one or more roots of the plant. The composition can be applied directly to the roots, e.g., by spraying or pouring onto the roots, and/or indirectly, e.g., by administering the composition to the soil in which the plant roots are growing (i.e., the rhizosphere). The composition can be applied to the seeds of the plant prior to or at the time of planting, or to any other part of the plant and/or its surrounding environment.

In certain embodiments, when, for example, the Fusarium fungi are too prolific in soil to be controlled entirely by the fungicidal composition, and/or, the Fusarium fungi have proliferated up into the plant through its vascular system, the method can further comprise applying the fungicidal composition with a biosurfactant composition.

Biosurfactants according to the subject invention include, for example, low-molecular-weight glycolipids, cellobiose lipids, lipopeptides, flavolipids, phospholipids, and high-molecular-weight polymers such as lipoproteins, lipopolysaccharide-protein complexes, and/or polysaccharide-protein-fatty acid complexes.

In one embodiment, the biosurfactants comprise glycolipids such as, for example, rhamnolipids (RLP), sophorolipids (SLP), trehalose lipids or mannosylerythritol lipids (MEL). In one embodiment, the biosurfactants comprise lipopeptides, such as, e.g., surfactin, iturin, fengycin, athrofactin, viscosin and/or lichenysin.

Advantageously, biosurfactants can supplement the antifungal capabilities of the fungicidal composition. Furthermore, due to the amphiphilic nature of biosurfactant molecules, they are capable of traveling up the plant's vascular system to reach fungi that have infected the above-ground parts of the plant.

In one embodiment, the method comprises applying the biosurfactant treatment composition to a plant and/or its surrounding environment at some point after the fungicidal composition has been applied. In some embodiments, depending upon the type of biosurfactant used in the biosurfactant composition, the biosurfactant(s) may kill the microorganisms of the fungicidal composition; however, by applying the biosurfactant to soil, it can supplement the fungicidal activity that the fungicidal composition has already performed. Furthermore, the biosurfactant composition can pass into the vascular system of an infected plant, traveling up the plant to control fungi present in, e.g., the xylem, phloem, trunk, branches, stems and foliage.

In one embodiment, the method comprises, either after, or simultaneously with, application of the fungicidal composition to soil and/or plant roots, applying the biosurfactant treatment directly to a part of the plant other than the roots. The biosurfactant composition can be applied directly to the inside of a plant, for example, into the vascular system (e.g., xylem and phloem) of the plant. Such direct application can comprise, for example, using a syringe to inject the biosurfactant treatment into, for example, the plant's trunk, branches, stems and/or foliage. Advantageously, this embodiment of the method allows for survival of the microorganisms present in the fungicidal soil treatment composition, as the biosurfactant treatment is not applied to the soil where those microorganisms are present.

In some embodiments, the biosurfactant treatment composition is applied to the plant and/or its environment without applying the fungicidal composition to the soil. In some embodiments, the fungicidal composition is applied to the soil without application of a biosurfactant treatment.

In some embodiments, the methods can be used for controlling Fusarium infections that are established in agricultural products (e.g., foliage, fruits and vegetables), either before, during, or after harvest. By applying a composition of the subject invention, for example, the fungicidal composition and/or the biosurfactant composition, to the agricultural products, the methods can reduce any further loss of the product due to an infection, including established infections.

In some embodiments, the methods can be used for reducing the population of pathogenic or deleterious microorganisms in the soil in which a plant grows, thereby allowing for re-colonization by beneficial microorganisms. In certain embodiments, the method comprises clearing the microbiome (niche) using a composition of the subject invention, followed by applying an enhancing agent for promoting beneficial microbe growth and/or directly inoculating the rhizosphere with one or more beneficial microorganisms. In one embodiment, the beneficial microorganisms are Pichia anomala, Pichia occidentalis, Trichoderma harzianum and/or Bacillus amyloliquefaciens.

Advantageously, the present invention can be used without releasing large quantities of inorganic compounds into the environment. Additionally, the compositions and methods utilize components that are biodegradable and toxicologically safe. Thus, the present invention can be used as a “green” pesticide.

Furthermore, the subject invention can be produced and used in remote locations, for example, in a banana grove. This allows for efficient delivery of fresh fermentation medium (e.g., broth) with active metabolites; a mixture of cells and fermentation medium; compositions with a high density of cells; and microbe-based products on short-order.

### BRIEF DESCRIPTION OF THE FIGURES

FIG. 1 shows inhibition of Fusarium oxysporum on a plate by sophorolipids (SLP).

FIG. 2 shows inhibition of F. oxysporum on a plate by Pichia anomala.

FIG. 3 shows F. oxysporum culture (untreated control) under a microscope. Hyphae were abundant.

FIG. 4A-B shows F. oxysporum culture treated with 1% SLP. Initial culture is shown in (4A). Hyphae were destroyed within three days (4B).

FIG. 5A-C shows F. oxysporum culture treated with P. anomala under the microscope after one day. Cells of P. anomala attached to the F. oxysporum hyphae (5A-B). The cells began to penetrate transparent, likely dead, hyphae (5C).

FIG. 6A-B shows F. oxysporum culture treated with P. anomala after day three of treatment. F. oxysporum hyphae appeared to be destroyed and/or dying.

FIG. 7A-B shows F. oxysporum culture treated with P. anomala after day seven. Most of the F. oxysporum hyphae appeared dead and destroyed.

FIG. 8A-B shows F. oxysporum culture treated with a P. anomala initially after day three of treatment. Similar effects were observed as with ordinary, non-mutated P. anomala, including destruction of F. oxysporum hyphae.

FIG. 9A-B shows F. oxysporum culture treated with P. occidentalis after the first day of treatment. The yeast cells were already actively attaching to the F. oxysporum hyphae and killing them directly, as well as inhibiting their growth. Some hyphae were thinned by the extracellular activity.

FIG. 10A-B shows the culture treated with P. occidentalis after day 3 of treatment. Only residual hyphae remained, with P. occidentalis cells growing in the location where the hyphae were previously, even repeating the structure of the hyphae. The culture of P. occidentalis after treatment was pure or nearly pure.

FIG. 11 shows an agar plate with the culture treated with P. occidentalis. No F. oxysporum was detected after day 2 and/or 3.

FIG. 12A-C shows three repeats of the agar plates of FIG. 1I. After three days, each of the three plates (A, B and C) showed the same results (no F. oxysporum detected).

### DETAILED DESCRIPTION OF THE INVENTION

The subject invention provides microbes, as well as by-products of their growth, such as biosurfactants. The subject invention also provides methods of using these microbes and their by-products, as well as methods and systems for producing them. Advantageously, the microbe-based products and methods of the subject invention are environmentally-friendly, non-toxic and cost-effective.

In preferred embodiments, the subject invention provides fungicidal compositions and methods of their use. Specifically, the subject invention provides for treatment and/or prevention of infection by Fusarium pathogens such as, e.g., Fusarium oxysporum, that infect, e.g., banana crops.

In one embodiment, a fungicidal composition is provided, comprising one or more yeast strains and/or growth by-products thereof. In preferred embodiments, the one or more yeast strains are selected from Pichia clade yeasts. Even more preferably, the yeast strains are selected from Pichia occidentalis and Pichia anomala (a.k.a. Wickerhamomyces anomalus).

The fungicidal composition can comprise fermentation medium and/or purified or unpurified growth by-products, such as biosurfactants, killer toxins, enzymes and/or other metabolites. The microbes can be live or inactive.

The composition is preferably formulated for application to soil, seeds, whole plants or plant parts. In preferred embodiments, the composition is formulated as a soil and/or root treatment.

In certain embodiments, methods for treating and/or preventing Fusarium infections in plants, wherein the fungicidal composition is contacted with a plant part. In a specific embodiment, the composition is contacted with one or more roots of the plant.

In certain embodiments, the method can further comprise applying the fungicidal composition with a biosurfactant composition.

In some embodiments, the biosurfactant treatment composition is applied to the plant and/or its environment without applying the fungicidal composition to the soil. In some embodiments, the fungicidal composition is applied to the soil without application of a biosurfactant treatment.

### Selected Definitions

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. In some embodiments, the microbes are present, with medium in which they were grown, in the microbe-based composition. The cells may be present at, for example, a concentration of 1×10⁴, 1×10⁵, 1×10⁶, 1×10⁷, 1×10⁸, 1×10⁹, 1×10¹⁰, 1×10¹¹, 1×10¹² or 1×10¹³ or more CFU per milliliter of the composition.

The subject invention further provides “microbe-based products,” which are products that are to be applied in practice to achieve a desired result. The microbe-based product can be simply the microbe-based composition harvested from the microbe cultivation process, or individual components thereof, such as supernatant. Alternatively, the microbe-based product may comprise further ingredients that have been added. These additional ingredients can include, for example, stabilizers, buffers, appropriate carriers, such as water, salt solutions, or any other appropriate carrier, added nutrients to support further microbial growth, non-nutrient growth enhancers, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, a “biofilm” is a complex aggregate of microorganisms, wherein the cells adhere to each other. In some embodiments, biofilms can adhere to surfaces. The cells in biofilms are physiologically distinct from planktonic cells of the same organism, which are single cells that can float or swim in liquid medium.

As used herein, an “isolated” or “purified” nucleic acid molecule, polynucleotide, polypeptide, protein or organic compound such as a small molecule, is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. An “isolated” microbial strain means that the strain is removed from the environment in which it exists in nature. Thus, the isolated strain may exist as, for example, a biologically pure culture, or as spores (or other forms of the strain) in association with a carrier.

As used here in, a “biologically pure culture” is one that has been isolated from materials with which it is associated in nature. In a preferred embodiment, the culture has been isolated from all other living cells. In further preferred embodiments, the biologically pure culture has advantages characteristics compared to a culture of the same microbe as it exists in nature. The advantageous characteristics can be, for example, enhanced probation of one or more by-products of their growth.

In certain embodiments, purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (e.g., a growth by-product) or a substance necessary for taking part in a particular metabolic process. A metabolite can be an organic compound that is a starting material (e.g., glucose), an intermediate (e.g., acetyl-CoA) in, or an end product (e.g., n-butanol) of metabolism. Examples of metabolites include, but are not limited to, biopolymers, enzymes, “killer toxins,” acids, solvents, alcohols, proteins, vitamins, minerals, microelements, amino acids, carbohydrates and biosurfactants.

Ranges provided herein are understood to be shorthand for all of the values within the range. For example, a range of 1 to 20 is understood to include any number, combination of numbers, or sub-range from the group consisting of 1, 2, 3, 4, 5, 6, 7, 8, 9, 10, 11, 12, 13, 14, 15, 16, 17, 18, 19, 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “non-pathogenic” means incapable of causing disease to an organism.

As used herein, “prevention” means avoiding, delaying, forestalling, or minimizing the onset or progression of a particular situation or occurrence. Prevention can include, but does not require, absolute or complete prevention, meaning the situation or occurrence may still develop, but at a later time than it would without preventative measures. Prevention can include reducing the severity of the onset of a situation or occurrence, and/or inhibiting the progression of the situation or occurrence to a more severe situation or occurrence.

As used herein, “reduce” refers to a negative alteration, and the term “increase” refers to a positive alteration, of at least 1%, 5%, 10%, 25%, 50%, 75%, or 100%.

As used herein, “surfactant” refers to a compound that lowers the surface tension (or interfacial tension) between two liquids or between a liquid and a solid. Surfactants act as, e.g., detergents, wetting agents, emulsifiers, foaming agents, and dispersants. A “biosurfactant” is a surfactant produced by a living organism.

As used herein, “agriculture” means the cultivation and breeding of plants, algae and/or fungi for food, fiber, biofuel, medicines, cosmetics, supplements, ornamental purposes and other uses. The products of agriculture can include, but are not limited to, fruits, vegetables, seeds, mushrooms, oils, tubers, foliage, and flowers that are harvested for such uses.

As used herein, “enhancing” means improving or increasing. For example, enhanced plant health means improving the plant's ability to grow and thrive, including the plant's ability to ward off pests and/or diseases, and the plant's ability to survive droughts and/or overwatering. Enhanced plant growth means increasing the size and/or mass of a plant, or improving the ability of the plant to reach a desired size and/or mass. Enhanced yields mean improving the end products produced by the plants in a crop, for example, by increasing the number of fruits per plant, increasing the size of the fruits, and/or improving the quality of the fruits (e.g., taste, texture).

As used herein, a “pest” is any organism, other than a human, that is destructive, deleterious and/or detrimental to humans or human concerns (e.g., agriculture, horticulture, livestock care, aquaculture). Pests may cause and/or carry infections, infestations and/or disease. Pests can cause direct harm to, for example, plants, by ingesting plant parts. Pests may be single- or multi-cellular organisms, including but not limited to, viruses, fungi, bacteria, parasites, arthropods and/or nematodes.

As used herein, the term “control” of a pest or an infection therewith extends to the act of killing, disabling, immobilizing, or reducing population numbers of a pest, or otherwise rendering the pest substantially incapable of causing harm. For example, the term “pesticidal” means capable of controlling a pest, and “fungicidal” means capable of controlling a fungal pest. A “pesticidally-effective” and/or “fungicidally-effective” amount of a substance is an amount that is capable of pesticidal/fungicidal action.

As used herein, “treatment” means the eradicating, reducing, ameliorating, reversing, or preventing of a degree, sign or symptom of a condition or disorder to any extent, and includes, but does not require, a complete cure of the condition or disorder. Treating can be curing, improving, or partially ameliorating a disorder. In some embodiments, treatment can comprise controlling a pest that causes an infection, infestation, or other disease.

As used herein, a “soil amendment” or a “soil conditioner” is any compound, material, or combination of compounds or materials that are added into soil to enhance the physical properties of the soil. Soil amendments can include organic and inorganic matter, and can further include, for example, fertilizers, pesticides and/or herbicides. Nutrient-rich, well-draining soil is essential for the growth and health of plants, and thus, soil amendments can be used for enhancing the growth and health of plants by altering the nutrient and moisture content of soil. Soil amendments can also be used for improving many different qualities of soil, including but not limited to, soil structure (e.g., preventing compaction); improving the nutrient concentration and storage capabilities; improving water retention in dry soils; and improving drainage in waterlogged soils.

As used herein, “environmental stressor” refers to an abiotic, or non-living, condition that has a negative impact on a living organism in a specific environment. The environmental stressor must influence the environment beyond its normal range of variation to adversely affect the population performance or individual physiology of the organism in a significant way. Examples of environmental stressors include, but are not limited to, drought, extreme temperatures, flood, high winds, natural disasters, soil pH changes, high radiation, compaction of soil, and others.

The description herein of any aspect or embodiment of the invention using terms such as “comprising,” “having,” “including” or “containing” with reference to an element or elements is intended to provide support for a similar aspect or embodiment of the invention that “consists of,” “consists essentially of,” or “substantially comprises” that particular element or elements, unless otherwise stated or clearly contradicted by context.

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references referred to or cited herein are incorporated by reference in their entirety, including all figures and tables, to the extent they are not inconsistent with the explicit teachings of this specification.

### Fungicidal Composition

In one embodiment, a fungicidal composition is provided, comprising one or more yeast strains and/or growth by-products thereof. In preferred embodiments, the one or more yeast strains are selected from Pichia Glade yeasts. Even more preferably, the yeast strains are selected from Pichia occidentalis and Pichia anomala (a.k.a. Wickerhamomyces anomalus).

The species and ratio of microorganisms and other ingredients in the composition can be customized according to, for example, the plant being treated, the soil type where the plant is growing, the health of the plant at the time of treatment, as well as other factors.

The microorganisms of the subject fungicidal compositions can be obtained through cultivation processes ranging from small to large scale. These cultivation processes include, but are not limited to, submerged cultivation/fermentation, solid state fermentation (SSF), and combinations thereof.

The fungicidal composition can comprise the leftover fermentation medium containing a live and/or an inactive culture, purified or unpurified microbial growth by-products, such as biosurfactants, killer toxins, enzymes and/or other metabolites, and/or any residual nutrients.

The composition may be, for example, at least, by weight, 1%, 5%, 10%, 25%, 50%, 75%, or 100% growth medium. The amount of biomass in the composition, by weight, may be, for example, anywhere from 0% to 100%, 10% to 75%, or 25% to 50%, inclusive of all percentages therebetween.

The product of fermentation may be used directly, with or without extraction or purification. If desired, extraction and purification can be easily achieved using standard extraction and/or purification methods or techniques described in the literature.

The microorganisms in the fungicidal composition may be in an active or inactive form, or in the form of vegetative cells and/or any other form of reproductive propagule.

In one embodiment, the different strains of microbe are grown separately and then mixed together to produce the fungicidal composition.

The composition is preferably formulated for application to soil, seeds, whole plants or plant parts (including, but not limited to, roots, tubers, stems, flowers, leaves and the vascular system). In certain embodiments, the composition is formulated as, for example, concentrated liquid, dust, granules, microgranules, pellets, wettable powder, flowable powder, emulsions, microcapsules, oils, or aerosols. In preferred embodiments, the composition is formulated as a soil treatment.

To improve or stabilize the effects of the composition, it can be blended with suitable adjuvants and then used as such, or after dilution, if necessary. In preferred embodiments, the composition is formulated as a liquid, or as dry powder or granules that can be mixed with water and other components to form a liquid product. In one embodiment, the composition can further comprise a source of protein and other nutrients, such as, for example, carbon, nitrogen, vitamins, micronutrients and amino acids, for enhanced growth of the beneficial microorganisms and production of fungicidal growth by-products.

The composition can be used either alone or in combination with other compounds for efficiently enhancing plant health, growth and/or yields. For example, in some embodiments, the composition can comprise additional components, such as herbicides, fertilizers, pesticides and/or soil amendments. In one embodiment, the composition can be mixed and/or applied with a commercial fertilizer, such as Scott's Miracle-Gro®, or another source of nutrients (e.g., nitrogen-phosphorous-potassium (NPK) macronutrients). Other additional components that can be included are macronutrients and/or micronutrients, such as magnesium, phosphate, nitrogen, potassium, selenium, calcium, sulfur, iron, copper, and zinc, and/or prebiotics, such as kelp extract, fulvic acid, fumaric acid, chitin (or chitin derivatives), humate, and/or humic acid. The exact materials and the quantities thereof can be customized to a particular crop or plant based on the needs thereof, and can be determined by a grower or an agricultural scientist.

The composition can also be used in combination with other crop management systems. In one embodiment, the composition can optionally comprise, or be applied with, natural and/or chemical pesticides and/or repellants, such as, for example, azoxystrobin, ipconazole, metalaxyl, trifloxystrobin, clothiandin, VOTiVO, thiamethoxam, cyantaniliprole, fludioxonil, tioxazafen, glycolipids, lipopeptides, deet, citronella, essential oils, mineral oils, garlic extract, chili extract, fatty acids, and/or any known commercial and/or homemade pesticide that is compatible with the combination of microorganisms being applied.

Further components can be added to the microbe-based composition, for example, buffering agents, carriers, other microbe-based compositions produced at the same or different facility, viscosity modifiers, preservatives, tracking agents, biocides, other microbes, surfactants, emulsifying agents, lubricants, solubility controlling agents, pH adjusting agents, preservatives, stabilizers and ultra-violet light resistant agents.

### Growth of Microorganisms

The subject invention utilizes methods for cultivation of microorganisms and production of microbial metabolites and/or other by-products of microbial growth. The subject invention further utilizes cultivation processes that are suitable for cultivation of microorganisms and production of microbial metabolites on a desired scale. These cultivation processes include, but are not limited to, submerged cultivation/fermentation, solid state fermentation (SSF), and modifications, hybrids and/or combinations thereof.

As used herein “fermentation” refers to cultivation or growth of cells under controlled conditions. The growth could be aerobic or anaerobic. In preferred embodiments, the microorganisms are grown using SSF and/or modified versions thereof.

In one embodiment, the subject invention provides materials and methods for the production of biomass (e.g., viable cellular material), extracellular metabolites (e.g. small molecules and excreted proteins), residual nutrients and/or intracellular components (e.g. enzymes and other proteins).

The microbe growth vessel used according to the subject invention can be any fermenter or cultivation reactor for industrial use. In one embodiment, the vessel is part of a distributed microbial growth facility. In one embodiment, the growth vessel is part of a portable fermentation system that can be operated in remote locations, for example, a rainforest or banana grove.

The vessel may have functional controls/sensors or may be connected to functional controls/sensors to measure important factors in the cultivation process, such as pH, oxygen, pressure, temperature, humidity, microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique. Dilution plating is a simple technique used to estimate the number of organisms in a sample. The technique can also provide an index by which different environments or treatments can be compared.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of liquid, and air spargers for supplying bubbles of gas to liquid for dissolution of oxygen into the liquid.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sesame oil, and/or linseed oil; etc. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, sodium chloride, calcium carbonate, and/or sodium carbonate. These inorganic salts may be used independently or in a combination of two or more.

In some embodiments, the method for cultivation may further comprise adding additional acids and/or antimicrobials in the medium before, and/or during the cultivation process. Antimicrobial agents or antibiotics are used for protecting the culture against contamination. Additionally, antifoaming agents may also be added to prevent the formation and/or accumulation of foam when gas is produced during submerged cultivation.

In one embodiment, the microorganisms can be grown on a solid or semi-solid substrate, such as, for example, corn, wheat, soybean, chickpeas, beans, oatmeal, pasta, rice, and/or flours or meals of any of these or other similar substances.

The pH of fermentation should be suitable for the microorganism of interest. Buffers, and pH regulators, such as carbonates and phosphates, may be used to stabilize pH near a preferred value. When metal ions are present in high concentrations, use of a chelating agent in the medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, the subject invention further provides a method for producing microbial metabolites such as, for example, biosurfactants, enzymes, proteins, ethanol, lactic acid, beta-glucan, peptides, metabolic intermediates, polyunsaturated fatty acid, and lipids, by cultivating a microbe strain of the subject invention under conditions appropriate for growth and metabolite production; and, optionally, purifying the metabolite. The metabolite content produced by the method can be, for example, at least 20%, 30%, 40%, 50%, 60%, 70%, 80%, or 90%.

In the case of submerged fermentation, the biomass content is, for example, from 5 g/l to 180 g/l, or more, or 10 g/l to 150 g/l.

The cell concentration may be, for example, 1×10⁹, 1×10¹⁰, 1×10¹¹, 1×10¹² or 1×10¹³ cells or spores per gram of final product.

The microbial growth by-product produced by microorganisms of interest may be retained in the microorganisms or secreted into the growth medium. The medium may contain compounds that stabilize the activity of microbial growth by-product.

The method and equipment for cultivation of microorganisms and production of the microbial by-products can be performed in a batch, a quasi-continuous process, or a continuous process.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells, spores, conidia, hyphae and/or mycelia remains in the vessel as an inoculant for a new cultivation batch. The composition that is removed can be a cell-free medium or contain cells, spores, or other reproductive propagules, and/or a combination of thereof. In this manner, a quasi-continuous system is created.

Advantageously, the method does not require complicated equipment or high energy consumption. The microorganisms of interest can be cultivated at small or large scale on site and utilized, even being still-mixed with their media.

Advantageously, the microbe-based products can be produced in remote locations. The microbe growth facilities may operate off the grid by utilizing, for example, solar, wind and/or hydroelectric power.

Microbial Strains Grown in Accordance with the Subject Invention

The microorganisms grown according to the systems and methods of the subject invention can be, for example, bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In one embodiment, the microorganism is a yeast or fungus. Yeast and fungus species suitable for use according to the current invention, include Aureobasidium (e.g., A. pullulans), Blakeslea, Candida (e.g., C. apicola, C. bombicola), Entomophthora, Saccharomyces (e.g., S. boulardii sequela, S. cerevisiae, S. torula), Issatchenkia, Mortierella, Mycorrhiza, Penicillium, Phycomyces, Pseudozyma (e.g., P. aphidis), Starmerella (e.g., S. bombicola), and/or Trichoderma (e.g., T. reesei, T. harzianum, T. hamatum, T. viride).

In certain embodiments, the microorganism is any yeast known as a “killer yeast” characterized by its secretion of toxic proteins or glycoproteins, to which the strain itself is immune. These can include, for example, Candida (e.g., C. nodaensis), Cryptococcus, Debaryomyces (e.g., D. hansenii), Hanseniaspora, (e.g., H. uvarum), Hansenula, Kluyveromyces (e.g., K phaffii), Pichia (e.g., P. anomala, P. guielliermondii, P. occidentalis, P. kudriavzevii), Saccharomyces (e.g., S. cerevisiae), Torulopsis, Ustilago (e.g., U maydis), Wickerhamomyces (e.g., W. anomalus), Williopsis (e.g., W. mrakii), Zygosaccharomyces (e.g., Z bailii), and others.

In a specific embodiment, the microbial strains are Pichia yeasts selected from Pichia occidentalis (and mutants thereof), Pichia anomala (and mutants thereof), and combinations thereof. Pichia occidentalis (a.k.a. Issatchenki occidentalis) is most often associated with fruit and natural fermentation of food products. Pichia anomala (a.k.a. Wickerhamomyces anomalus) is an effective producer of various solvents (e.g., ethyl acetate), enzymes (e.g., phytase), killer toxins (e.g., exo-β-1,3-glucanase), as well as glycolipid biosurfactants.

In one embodiment, the microorganism is a spore-producing Trichoderma spp. fungus. In a specific embodiment, the microorganism is Trichoderma harzianum or mutants thereof.

In one embodiment, the microorganisms are bacteria, including gram-positive and gram-negative bacteria. The bacteria may be, for example Bacillus (e.g., B. subtilis, B. licheniformis, B. firmus, B. laterosporus, B. megaterim, B. amyloliquifaciens), Clostridium (C. butyricum, C. tyrobutyricum, C. acetobutyricum, Clostridium NIPER 7, and C. beijerinckii), Azobacter (A. vinelandii, A. chroococcum), Pseudomonas (P. chlororaphis subsp. aureofaciens (Kluyver), P. aeruginosa), Agrobacterium radiobacter, Azospirillumbrasiliensis, Rhizobium, Sphingomonas paucimobilis, Ralslonia eulropha, and/or Rhodospirillum rubrum.

In one embodiment, the microorganism is a species of Bacillus, for example, a strain of B. subtilis or a mutant thereof, or a strain of B. amyloliquefaciens (e.g., B. amyloliquefaciens subsp. locus) or a mutant thereof.

In one embodiment, the microorganisms in the subject microbe-based composition work synergistically with one another to control Fusarium fungi and enhance plant health, growth and/or yields.

### Preparation of Microbe-Based Products

One microbe-based product (e.g., a soil treatment composition) of the subject invention is simply the fermentation medium containing the microorganisms and/or the microbial metabolites produced by the microorganisms and/or any residual nutrients. The product of fermentation may be used directly without extraction or purification. If desired, extraction and purification can be easily achieved using standard extraction and/or purification methods or techniques described in the literature.

The microorganisms in the microbe-based products may be in an active or inactive form, or in the form of vegetative cells, reproductive spores, conidia, mycelia, hyphae, or any other form of microbial propagule. The microbe-based products may also contain a combination of any of these forms of a microorganism.

In one embodiment, the different strains of microbe are grown separately and then mixed together to produce the microbe-based product. The microbes can, optionally, be blended with the medium in which they are grown and dried prior to mixing.

The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

Upon harvesting the microbe-based composition from the growth vessels, further components can be added as the harvested product is placed into containers or otherwise transported for use. The additives can be, for example, buffers, carriers, other microbe-based compositions produced at the same or different facility, viscosity modifiers, chelating agents, adherents, preservatives, nutrients for microbe growth (e.g., prebiotics), surfactants, emulsifying agents, lubricants, solubility controlling agents, tracking agents, solvents, biocides, antibiotics, pH adjusting agents, stabilizers, ultra-violet light resistant agents, fatty acids (or their salts or derivatives), other microbes and other suitable additives that are customarily used for such preparations.

In one embodiment, the composition may further comprise buffering agents including organic and amino acids or their salts. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

The pH of the microbe-based product should be suitable for the microorganism of interest as well as for the plant being treated. In a preferred embodiment, the pH of the final microbe-based composition ranges from 5.5-7.5.

In a further embodiment, pH adjusting agents include potassium hydroxide, ammonium hydroxide, potassium carbonate or bicarbonate, hydrochloric acid, nitric acid, sulfuric acid or a mixture.

In one embodiment, prebiotics can be added to the microbe-based product, including, for example, kelp extract, fulvic acid, humate and/or humic acid.

In one embodiment, additional components such as sodium bicarbonate or carbonate, sodium sulfate, sodium phosphate, sodium biphosphate, can be included in the formulation.

In one embodiment, glucose and glycerin can be added to the microbe-based product to serve as an osmoticum during storage and transport. In one embodiment, molasses can be included.

Optionally, the composition can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C.

The microbe-based compositions may be used without further stabilization, preservation, and storage, however. Advantageously, direct usage of these microbe-based compositions preserves a high viability of the microorganisms, reduces the possibility of contamination from foreign agents and undesirable microorganisms, and maintains the activity of the by-products of microbial growth.

In other embodiments, the composition (microbes, growth medium, or microbes and medium) can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 pint to 1,000 gallons or more. In certain embodiments the containers are 1 gallon, 2 gallons, 5 gallons, 25 gallons, or larger.

### Methods of Treating a Plant Fungal Infection

In one embodiment, methods are provided for controlling Fusarium fungi in or on a plant and/or a plant's surrounding environment, wherein one or more non-pathogenic microorganisms, and/or their growth by-products, are contacted with the plant and/or the surrounding environment. In certain embodiments, the Fusarium infection is established, or already exists, in or on the plant and/or surrounding environment. Advantageously, the compositions can be applied to non-dormant plant tissue without causing phytotoxicity.

The method can comprise contacting a fungicidal composition of the subject invention, comprising one or more microorganisms and/or a growth by-product thereof, with the plant and/or its surrounding environment. Preferably, the one or more microorganisms and/or their growth by-products are capable of fungicidal action. In one embodiment, the one or more fungicidal microorganisms are selected from Pichia yeasts, for example, P. occidentalis and P. anomala (a.k.a. Wickerhamomyces anomalus), as well as mutants thereof.

In one embodiment, the microorganisms are further selected from Trichoderma harzianum and Bacillus amyloliquefaciens.

Advantageously, the subject method can even be used to enhance health, growth and/or yields in plants having compromised immune health due to an infection from a Fusarium fungus or from an environmental stressor. Furthermore, the subject method can be used to reduce the amount of plant and/or crop loss due to plant damage and/or death caused by Fusarium infections. Even further, the subject methods can be used for controlling fungal infections in or on plant tissue without phytotoxicity.

In one embodiment, the method can be used for treating any plant species that is susceptible to infection by a Fusarium fungus. In specific embodiments, the method can be used for treating a plant from the Musa genus, such as a banana or a plantain. In certain embodiments, the plant receiving treatment is healthy, i.e., not infected with a pathogenic disease. In other embodiments, the plant is affected by an established, or existing, plant disease. The disease is preferably a fungal disease.

In preferred embodiments, the subject method can be used for controlling Fusarium fungi, such as, for example, F. avenaceum, F. bubigeum, F. culmorum, F. graminearum, F. langsethiae, F. oxysporum, F. proliferatum, F. sporotrichioides, F. poae, F. reseum, F. solani, F. tricinctum, F. verticillioides, F. virguliforme, F. xylariodides and any other Fusarium fungi that can infect plants.

In specific preferred embodiments, the subject method can be used to control Fusarium oxysporum, and/or Fusarium oxysporum f sp. cubense.

In certain embodiments, the method is effective at controlling hyphae of Fusarium spp. fungi. In certain embodiments, the method is effective at controlling spores of Fusarium spp. fungi.

The subject method can also be used to prevent and/or treat plant conditions and/or diseases, such as, for example, wilt, blight, rot and decay caused by Fusarium. Fusarium oxysporum is responsible for “Panama disease” as well as other types of Fusarium Wilt which commonly affect a wide variety of herbaceous plants, including: Musa, tomato, tobacco, legumes, cucurbits, and sweet potatoes.

Fusarium xylariodides is known to cause Coffee wilt disease (CWD), which causes, inter cilia, abscission of leaves, blackening of branches, and rotting of roots in Coffea spp., including, for example, C. liberica, C. canephora, and C. arabica.

Fusarium graminearum, Fusarium avenaceum, Fusarium culmorum, Fusarium sporotrichioides and Fusarium poae are all known to cause Fusarium Head Blight (also known as Fusarium Ear Blight), which is a common disease that affects cereals, such as wheat, barley, oats, rye, and triticale.

Fusarium solani causes a host plants' roots to rot, and is known to infect various plants and crops, including, for example, soybeans, avocado, citrus, orchids, passion fruit, peas, peppers, potato, and squash. Fusarium verticillioides is known to infect maize, causing seedling decay, stalk rot, and ear rot. Fusarium proliferatum is known to cause rot in, for example, garlic, asparagus and orchids. Fusarium roseum and Fusarium tricinctum are known to cause blight in turfgrasses, such as bentgrass, bluegrass and fescues.

The methods can comprise applying nutrients to enhance the growth of the one or more fungicidal microorganisms and/or production of fungicidal growth by-products. Such nutrients can include, for example, sources of carbon, nitrogen, potassium, phosphorus, magnesium, proteins, micronutrients, vitamins and/or amino acids.

As used herein, “applying” a composition or product, or “treating” an environment refers to contacting a composition or product with a target or site such that the composition or product can have an effect on that target or site. The effect can be due to, for example, microbial growth and/or the action of a metabolite, enzyme, biosurfactant or other growth by-product.

Application can further include contacting the microbe-based product directly with a plant, plant part, and/or the plant's surrounding environment (e.g., the soil). The microbe-product can be applied as a seed treatment or to the soil surface, or to the surface of a plant or plant part (e.g., to the surface of the roots, tubers, stems, flowers, leaves, fruit, or flowers). It can be sprayed as a liquid or a dry powder, dust, granules, microgranules, pellets, wettable powder, flowable powder, emulsions, microcapsules, oils, gels, pastes or aerosols.

To improve or stabilize the effects of the composition, it can be blended with suitable adjuvants and then used as such or after dilution if necessary. In preferred embodiments, the composition is formulated as a dry powder, which can be mixed with water and other components to form a liquid product. In one embodiment, the composition can comprise glucose, in addition to an osmoticum substance, to ensure optimum osmotic pressure during storage and transport of the dry product. In one embodiment, the osmoticum substance can be glycerin.

In certain embodiments, the fungicidal composition is contacted with a plant part. In a specific embodiment, the composition is contacted with one or more roots of the plant. The composition can be applied directly to the roots, e.g., by spraying or pouring onto the roots, and/or indirectly, e.g., by administering the composition to the soil in which the plant roots are growing (i.e., the rhizosphere). The composition can be applied to the seeds of the plant prior to or at the time of planting, or to any other part of the plant and/or its surrounding environment.

In one embodiment, the composition is injected into a plant using, for example, a syringe. The composition can be injected into, for example, the roots, trunk, stems and/or vascular system of the plant if direct internal treatment of the plant is desired.

In one embodiment, wherein the method is used in a large scale setting, the method can comprise administering the fungicidal composition into a tank connected to an irrigation system used for supplying water, fertilizers, or other liquid compositions to a crop, orchard or field. Thus, the plant and/or soil surrounding the plant can be treated with the fungicidal composition via, for example, soil injection, soil drenching, or using a center pivot irrigation system, or with a spray over the seed furrow, or with sprinklers or drip irrigators. Advantageously, the method is suitable for treating hundreds of acres of a plantation, crop, orchard or field at one time.

In one embodiment, wherein the method is used in a smaller scale setting, such as in a home garden or greenhouse, the method can comprise pouring the fungicidal composition into the tank of a handheld lawn and garden sprayer having water and optionally other pesticides and nutrient sources therein, and spraying a plant and/or its surrounding environment with the mixture.

Plants and/or their environments can be treated at any point during the process of cultivating the plant. For example, the fungicidal composition can be applied to the soil prior to, concurrently with, or after the time when seeds are planted therein. It can also be applied at any point thereafter during the development and growth of the plant, including when the plant is flowering, fruiting, and during and/or after abscission of leaves.

In certain embodiments, the fungicidal compositions provided herein are applied to the soil surface without mechanical incorporation. The beneficial effect of the soil application can be activated by rainfall, sprinkler, flood, or drip irrigation, and subsequently delivered to, for example, the roots of plants to influence the root microbiome or facilitate uptake of the microbial product into the vascular system of the crop or plant to which the microbial product is applied. In an exemplary embodiment, the fungicidal compositions provided herein can be efficiently applied via a center pivot irrigation system or with a spray over the seed furrow.

In one embodiment, the method can be used for enhancing penetration of beneficial molecules through the outer layers of root cells.

In certain embodiments, when, for example, the Fusarium fungi are too prolific in soil to be controlled entirely by the fungicidal composition, and/or, the Fusarium fungi have proliferated up into the plant through its vascular system, the method can further comprise applying the fungicidal composition with a biosurfactant composition.

Biosurfactants are a structurally diverse group of surface-active substances produced by microorganisms. Biosurfactants are amphiphiles. They consist of two parts: a polar (hydrophilic) moiety and non-polar (hydrophobic) group. Due to their amphiphilic structure, biosurfactants increase the surface area of hydrophobic water-insoluble substances and increase the water bioavailability of such substances. Additionally, biosurfactants accumulate at interfaces, thus reducing interfacial tension and leading to the formation of aggregated micellar structures in solution. The ability of biosurfactants to form pores and destabilize biological membranes permits their use as, e.g., antibacterial and antifungal agents.

Furthermore, biosurfactants can inhibit adhesion of undesirable microorganisms to a variety of surfaces, prevent the formation of biofilms, and can have powerful emulsifying and demulsifying properties. Even further, biosurfactants can also be used to improve wettability and to achieve even solubilization and/or distribution of fertilizers, nutrients, and water in the soil.

Advantageously, biosurfactants are biodegradable and can be efficiently produced, according to the subject invention, using selected organisms on renewable substrates. Most biosurfactant-producing organisms produce biosurfactants in response to the presence of a hydrocarbon source (e.g. oils, sugar, glycerol, etc.) in the growing media. Other media components such as concentration of iron can also affect biosurfactant production significantly.

Microbial biosurfactants are produced by a variety of microorganisms such as bacteria, fungi, and yeasts. Exemplary biosurfactant-producing microorganisms include Starmerella spp. (e.g., S. bombicola), Pseudomonas spp. (e.g., P. aeruginosa, P. putida, P. florescens, P. Tragi, P. syringae); Flavobacterium spp.; Bacillus spp. (e.g., B. subtilis, B. amyloliquefaciens, B. pumillus, B. cereus, B. licheniformis); Wickerhamomyces spp. (e.g., W. anomalus), Candida spp. (e.g., C. albicans, C. rugosa, C. tropicalis, C. lipolytica, C. torulopsis); Saccharomyces (e.g., S. cerevisiae); Pseudozyma spp. (e.g., P. aphidis); Rhodococcus spp. (e.g., R. erythropolis); Arthrobacter spp.; Campylobacter spp.; Cornybacterium spp.; as well as others.

Biosurfactants according to the subject invention include, for example, low-molecular-weight glycolipids, cellobiose lipids, lipopeptides, flavolipids, phospholipids, and high-molecular-weight polymers such as lipoproteins, lipopolysaccharide-protein complexes, and/or polysaccharide-protein-fatty acid complexes.

The hydrocarbon chain of a fatty acid acts as the common lipophilic moiety of a biosurfactant molecule, whereas the hydrophilic part is formed by ester or alcohol groups of neutral lipids, by the carboxylate group of fatty acids or amino acids (or peptides), organic acid in the case of flavolipids, or, in the case of glycolipids, by the carbohydrate.

In one embodiment, the biosurfactants according to the subject compositions comprise glycolipids and/or glycolipid-like biosurfactants, such as, for example, rhamnolipids (RLP), sophorolipids (SLP), trehalose lipids or mannosylerythritol lipids (MEL). In one embodiment, the biosurfactants comprise lipopeptides and/or lipopeptide-like biosurfactants, such as, e.g., surfactin, iturin, fengycin, athrofactin, viscosin and/or lichenysin. In certain embodiments, the biosurfactants are cardiolipins or cellobiose lipids.

In certain embodiments, the biosurfactant composition comprises more than one type of biosurfactant. In some embodiments, the total concentration of the biosurfactant composition is about 0.001 to about 5.0%, or about 0.005% to about 1.0%, or about 0.01% to about 0.1%, or about 0.05%.

In one embodiment, the biosurfactant composition comprises one or more glycolipid biosurfactants. In preferred embodiments, the biosurfactant composition comprises sophorolipids (SLP) in a concentration of 0.001% to 10%, 0.01% to 5%, 0.05% to 2%, and/or from 0.1% to 1%. In one embodiment, the biosurfactants are in purified form.

Advantageously, biosurfactants can supplement the antifungal capabilities of the fungicidal composition. Furthermore, due to, for example, the amphiphilic nature of biosurfactant molecules, they are capable of traveling up the plant's vascular system to reach fungi that have infected the above-ground parts of the plant. The biosurfactant composition can be applied continuously, as a single treatment, or as a plurality of serial treatments with limited time between each.

In one embodiment, the method comprises applying the biosurfactant treatment composition to a plant and/or its surrounding environment at some point after the fungicidal composition has been applied. In some embodiments, depending upon the type of biosurfactant used in the biosurfactant composition, the biosurfactant(s) may kill the microorganisms of the fungicidal composition; however, by applying the biosurfactant to soil, it can supplement the fungicidal activity that the fungicidal composition has already performed. Furthermore, the biosurfactant composition can pass into the vascular system of an infected plant, traveling up the plant to control fungi present in, e.g., the xylem, phloem, trunk, branches, stems and foliage.

In one embodiment, the biosurfactant treatment is applied in such a way that it does not contact the yeasts of the fungicidal composition. For example, in one embodiment, the biosurfactant composition is applied directly to a part of the plant other than the roots. The biosurfactant composition can be applied directly to the inside of a plant, for example, into the vascular system (xylem and phloem) of the plant. Direct application according to this embodiment can comprise, for example, using a syringe to inject the biosurfactant treatment into, for example, the plant's trunk, branches, stems and/or foliage. For trunks and/or stems of trees and larger plants, it may be necessary to drill a small hole into the trunk or stem to insert the syringe.

Advantageously, this embodiment of the method allows for survival of the microorganisms present in the fungicidal soil treatment composition, as the biosurfactant treatment is not applied to the soil where those microorganisms are present. Furthermore, injecting the treatment straight into a plant's circulatory system allows the compositions to dissipate rapidly throughout the plant while minimizing the amount of composition needed.

In some embodiments, the biosurfactant treatment composition is applied to the plant and/or its environment without applying the fungicidal composition to the soil. In some embodiments, the fungicidal composition is applied to the soil without application of a biosurfactant treatment.

In addition to treating and/or preventing fungal infections, the present invention can be used to enhance health, growth and yields of, for example, Musa crops, as well as any other plant or crop plant that is susceptible to and/or infected by a Fusarium fungal disease.

In certain embodiments, the methods and compositions according to the subject invention reduce damage to a plant caused by Fusarium by about 5%, 10%, 20%, 30%, 40%, 50%, 60% 70%, 80%, or 90% or more, compared to plants growing in an untreated environment.

In certain embodiments, the methods and compositions according to the subject invention lead to an increase in crop yield by about 5%, 10%, 20%, 30%, 40%, 50%, 60% 70%, 80%, or 90% or more, compared to untreated crops.

In one embodiment, the methods of the subject invention lead to a reduction in the amount of Fusarium in or on a plant or in a plant's surrounding environment by about 5%, 10%, 20%, 30%, 40%, 50%, 60% 70%, 80%, or 90% or more, compared to a plant growing in an untreated environment.

In one embodiment, the methods of the subject invention lead to an increase in the mass of a plant and/or a plant's fruit by about 5%, 10%, 20%, 30%, 40%, 50%, 60% 70%, 80%, or 90% or more, compared to a plant growing in an untreated environment.

The subject invention can also be used as a “niche-clearing” agent. In one embodiment, the fungicidal composition, and/or the biosurfactant composition, can be used to disrupt the existing balance of microorganisms present in the soil in which a plant is growing.

In certain embodiments, the soil microbiome in which a plant is growing comprises deleterious microbes, such as, for example, Fusarium spp. fungi. By clearing out or reducing the soil microbiome population, the subject methods provide for re-colonization of the rhizosphere with one or more beneficial microorganisms, which, in certain embodiments, can ward off and/or out-compete any deleterious species that may try to colonize or re-colonize.

Thus, in some embodiments, the method comprises clearing the soil microbiome using a composition of the subject invention, followed by applying an enhancing agent for promoting beneficial microbe growth and/or directly inoculating the rhizosphere with one or more beneficial microorganisms.

In one embodiment, the beneficial microorganisms are, for example, Pichia anomala, Pichia occidentalis, Trichoderma harzianum, Bacillus amyloliquefaciens, Azotobacter vinelandii, Frateuria aurantia and/or others known to be beneficial to a soil microbiome.

The subject invention can also be used to improve a variety of qualities in any type of soil, for example, clay, sandy, silty, peaty, chalky, loam soil, and/or combinations thereof. Furthermore, the methods and compositions can be used for improving the quality of dry, waterlogged, porous, depleted, compacted soils and/or combinations thereof.

In one embodiment, the method can be used for improving the drainage and/or dispersal of water in waterlogged soils. In one embodiment, the method can be used for improving water retention in dry soil. In one embodiment, the method can be used for improving nutrient retention in porous and/or depleted soils.

In one embodiment, the method controls pathogenic fungi themselves. In one embodiment, the method works by enhancing the immune health of plants to increase the ability to fight off infections.

In yet another embodiment, the method controls any pests that might act as vectors or carriers for pathogenic fungi, for example, insects, such as flies, aphids, ants, beetles, whiteflies, etc., that land on the plant and come in contact with the pathogen. Thus, the subject methods can prevent the spread of plant pathogenic fungi by controlling, i.e., killing, these carrier pests.

The method can be used either alone or in combination with application of other compounds for efficient enhancement of plant immunity, health, growth and/or yields, as well as other compounds for efficient treatment and prevention of plant pathogenic pests. For example, commercial and/or natural fertilizers, antibiotics, pesticides, herbicides and/or soil amendments can be applied alongside the compositions of the subject invention. In one embodiment, the method comprises applying fatty acid compositions alongside the subject compositions, including, for example, unsubstituted or substituted, saturated or unsaturated fatty acids, and/or salts or derivatives thereof.

In certain embodiments, the microbe-based products can be used to enhance the effectiveness of the other compounds, for example, by enhancing the penetration of a drug compound into a plant or pest. The microbe-based products can also be used to supplement other treatments, for example, antifungal treatments.

### Target Plants

The subject invention can be useful in the prevention and/or treatment of fungal infections that cause diseases in plants. In preferred embodiments, the fungal infection is caused by a Fusarium fungi.

As used here, the term “plant” includes, but is not limited to, any species of woody, ornamental or decorative, crop or cereal, fruit plant or vegetable plant, flower or tree, macroalga or microalga, phytoplankton and photosynthetic algae (e.g., green algae Chlamydomonas reinhardtii). “Plant” also includes a unicellular plant (e.g. microalga) and a plurality of plant cells that are largely differentiated into a colony (e.g. volvox) or a structure or tissue that is present at any stage of a plant's development. Such structures/tissues include, but are not limited to, a fruit, a seed, a shoot, a stem, a leaf, a root, a flower petal, etc. Plants can be standing alone, for example, in a garden, or can be one of many plants, for example, as part of an orchard, crop or pasture.

Types of plants that can benefit from application of the products and methods of the subject invention include, but are not limited to: row crops (e.g., corn, soy, sorghum, peanuts, potatoes, etc.), field crops (e.g., alfalfa, wheat, grains, etc.), tree crops (e.g., walnuts, almonds, pecans, hazelnuts, pistachios, etc.), citrus crops (e.g., orange, lemon, grapefruit, etc.), fruit crops (e.g., apples, pears, strawberries, blueberries, blackberries, etc.), turf crops (e.g., sod), ornamentals crops (e.g., flowers, vines, etc.), vegetables (e.g., tomatoes, carrots, etc.), vine crops (e.g., grapes, etc.), forestry (e.g., pine, spruce, eucalyptus, poplar, etc.), managed pastures (any mix of plants used to support grazing animals) and others.

In preferred embodiments, the crop plant is a banana plant, and/or a plant from the Musa genus. As used herein, the term “banana” generally refers to a plant bearing the type of fruit commonly referred to as a banana, belonging to the genus Musa.

Types of bananas which are suitable for use with the present invention include, but are not limited to, baby, manzano, burro, plantain, red, apple banana, Cavendish, lady finger, pisang, williams, and cooking. Additional examples of Musa plants according to the subject invention include, but are not limited to M acuminata (AA, AAA and AAAA groups); Musa x paradisiaca (AAAB, AAB, AABB, AB, ABB, and ABBB groups); Musa balbisiana (BB and BBB groups); Chingan, Lacatan, Lady Finger, Sugar, Pisang jari buaya, Senorita, Sinwobogi, Cavendish, Dwarf Cavendish, Grand Nain, Red Dacca, Dwarf Red, Gros Michel, East African Highlands, Bodies Altafort, Golden Beauty, Atan, Goldfinger Iholena, Maqueno, Popoulu, Mysore, Pisang Raja, Pome, Prata-ana, Latundan (silk, apple bananas), Pisang Seribu, Plu, Kalamagol, Pisang Awak, Ney Poovan, Blue Java, Bluggoe, Silver Bluggoe, Pelipita, Saba, Carbada, Benedetta, Tiparot, and Kluai Lep Chang Kut bananas; and plantains including, e.g., French plantain, Green French banana, Horn plantain, Nendran banana, Pink French banana, and Tiger banana.

In specific preferred embodiments, the plant is a Cavendish banana (Musa acuminata, AAA group).

Other example of plants for which the subject invention is useful include, but are not limited to, cereals and grasses (e.g., wheat, barley, rye, oats, rice, maize, sorghum, corn, turf), beets (e.g., sugar or fodder beets); fruit (e.g., grapes, strawberries, raspberries, blackberries, pomaceous fruit, stone fruit, soft fruit, apples, pears, plums, peaches, almonds, cherries or berries); leguminous crops (e.g., beans, lentils, peas or soya); oil crops (e.g., oilseed rape, mustard, poppies, olives, sunflowers, coconut, castor, cocoa or ground nuts); cucurbits (e.g., pumpkins, cucumbers, squash or melons); fiber plants (e.g., cotton, flax, hemp or jute); citrus fruit (e.g., oranges, lemons, grapefruit or tangerines); vegetables (e.g., spinach, lettuce, asparagus, cabbages, carrots, onions, tomatoes, potatoes or bell peppers); Lauraceae (e.g., avocado, Cinnamonium or camphor); and also tobacco, nuts, herbs, spices, medicinal plants, coffee, eggplants, sugarcane, tea, pepper, grapevines, hops, latex plants, cut flowers and ornamentals.

Further plants that can benefit from the products and methods of the invention include all plants that belong to the superfamily Viridiplantae, in particular monocotyledonous and dicotyledonous plants including fodder or forage legumes, ornamental plants, food crops, trees or shrubs selected from Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g., A. sativa, A. fatua, A. byzantina, A. fatua var. sativa, A. hybrida), Averrhoa carambola, Bambusa sp., Benincasa hispida, Bertholletia excelsea, Beta vulgaris, Brassica spp. (e.g., B. napus, B. rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Casktnea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g., E. guineensis, E. oleifera), Eleusine coracana, Eragrostis tef; Erianthus sp., Eriobotrya japonica, Eucalyptus sp., Eugenia un flora, Fagopyrum spp., Fagus spp., Festuca arundinacea, Ficus carica, Fortunella spp., Fragaria spp., Ginkgo biloba, Glycine spp. (e.g., G. max, Soja hispida or Sofa max), Gossypium hirsutum, Helianthus spp. (e.g., H annuus), Hemerocallis fulva, Hibiscus spp., Hordeum spp. (e.g., H vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g., L. esculentum, L. lycopersicum, L. pyriforms), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g., O. sativa, O. latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g., S. tuberosum, S. integrifolium or S. lycopersicum), Sorghum bicolor, Spinacia spp., Syzygium spp., Tagetes spp., Tamarindus indica, Theobroma cacao, Trifolium spp., Tripsacum dactyloides, Triticosecale rirnpaui, Triticum spp. (e.g., T. aestivum, T durum, T turgidum, T. hybernum, T macha, T sativum, T monococcum or T vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

Further examples of plants of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticurn aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Alacrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the embodiments include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Plants of the embodiments include crop plants (for example, corn, alfalfa, sunflower, Brassica, soybean, cotton, safflower, peanut, sorghum, wheat, millet, tobacco, etc.), such as corn and soybean plants.

Turfgrasses include, but are not limited to: annual bluegrass (Poa annua); annual ryegrass (Lolium multiflorum); Canada bluegrass (Poa compressa); Chewings fescue (Festuca rubra); colonial bentgrass (Agrostis tenuis); creeping bentgrass (Agrostis palustris); crested wheatgrass (Agropyron desertorum); fairway wheatgrass (Agropyron cristatum); hard fescue (Festuca longifolia); Kentucky bluegrass (Poa pratensis); orchardgrass (Dactylis glomerate); perennial ryegrass (Lolium perenne); red fescue (Festuca rubra); redtop (Agrostis alba); rough bluegrass (Poa trivialis); sheep fescue (Festuca ovine); smooth bromegrass (Bromus inermis); tall fescue (Festuca arundinacea); timothy (Phleum pretense); velvet bentgrass (Agrostis canine); weeping alkaligrass (Puccinellia distans); western wheatgrass (Agropyron smithii); Bermuda grass (Cynodon spp.); St. Augustine grass (Stenotaphrum secundatum); zoysia grass (Zoysia spp.); Bahia grass (Paspalum notatum); carpet grass (Axonopus affinis); centipede grass (Eremochloa ophiuroides); kikuyu grass (Pennisetum clandesinum); seashore paspalum (Paspalum vaginatum); blue gramma (Bouteloua gracilis); buffalo grass (Buchloe dactyloids); sideoats gramma (Bouteloua curtipendula).

Additional plants of interest include grain plants that provide seeds of interest, oil-seed plants, and leguminous plants. Seeds of interest include grain seeds, such as corn, wheat, barley, rice, sorghum, rye, millet, etc. Oil-seed plants include cotton, soybean, safflower, sunflower, Brassica, maize, alfalfa, palm, coconut, flax, castor, olive etc. Leguminous plants include beans and peas. Beans include guar, locust bean, fenugreek, soybean, garden beans, cowpea, mungbean, lima bean, fava bean, lentils, chickpea, etc. Cannabis (e.g., sativa, indica, and ruderalis) and industrial hemp are also envisioned.

All plants and plant parts can be treated in accordance with the invention. In this context, plants are understood as meaning all plants and plant populations such as desired and undesired wild plants or crop plants (including naturally occurring crop plants). Crop plants can be plants that can be obtained by traditional breeding and optimization methods or by biotechnological and recombinant methods, or combinations of these methods, including the transgenic plants and the plant varieties.

Plant parts are understood as meaning all aerial and subterranean parts and organs of the plants such as shoot, leaf, flower and root, examples which may be mentioned being leaves, needles, stalks, stems, flowers, fruit bodies, fruits and seeds, but also roots, tubers and rhizomes. The plant parts also include crop material and vegetative and generative propagation material, for example cuttings, tubers, rhizomes, slips and seeds. Furthermore, plant parts can be dormant and/or non-dormant, where non-dormant tissues include growing vegetation and fruits. Advantageously, the compositions of the subject invention can be applied to non-dormant tissue without phytotoxicity.

In one embodiment, the subject invention is particularly useful for the control of Fusarium infections on agricultural products (e.g., foliage, fruits and vegetables, both pre- and post-harvest). An infection can become established on agricultural products, for example, during growth, harvesting, storage, packing and shipping.

One example of controlling post-harvest spoilage of agricultural products pertains to Fusarium infections of harvested corn (Fusarium Ear Rot, caused by, e.g., F. verticillioides). Fusarium ear rot can enter ears of corn through wounds from insects or other human or environmental damage, causing individual kernels to turn brown, and sometimes, entire ears to be consumed. Furthermore, mycotoxins produced by some species of Fusarium can be toxic to some mammals, making the corn inedible.

By treating agricultural products, e.g., corn, with a composition according to the subject invention, an infection can be controlled, and further spread of the infection to other plants and/or agricultural products can be prevented during transport and processing. In these cases, where eradication of established infections is achieved in an agricultural product, the further protection of agricultural products from subsequent infections can be achieved by applying, simultaneously or subsequently, an additional fungicidal or other protective compound, for example, a wax or finish, to the agricultural product.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the subject invention can be located at the location where the microbe-based product will be used (e.g., a banana grove). For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. This allows for a scaled-down bioreactor (e.g., smaller fermentation vessel, smaller supplies of starter material, nutrients and pH control agents), which makes the system efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules, or a mixture of vegetative cells and propagules.

In one embodiment, the microbe growth facility is located on, or near, a site where the microbe-based products will be used (e.g., a banana grove), for example, within 300 miles, 200 miles, or even within 100 miles. In another embodiment, the microbe growth facility is portable for ease of transport between application locations, even those in remote, hard-to-traverse areas (e.g., a rainforest).

Advantageously, this allows for the compositions to be tailored for use at a specified location. The formula and potency of microbe-based compositions can be customized for specific local conditions at the time of application, such as, for example, which soil type, plant and/or crop is being treated; what season, climate and/or time of year it is when a composition is being applied; and what mode and/or rate of application is being utilized.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

Furthermore, by producing a composition locally, the formulation and potency can be adjusted in real time to a specific location and the conditions present at the time of application. This provides advantages over compositions that are pre-made in a central location and have, for example, set ratios and formulations that may not be optimal for a given location.

The microbe growth facilities provide manufacturing versatility by their ability to tailor the microbe-based products to improve synergies with destination geographies. Advantageously, in preferred embodiments, the systems of the subject invention harness the power of naturally-occurring local microorganisms and their metabolic by-products.

The cultivation time for the individual vessels may be, for example, from 1 to 7 days or longer. The cultivation product can be harvested in any of a number of different ways.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Solid State Fermentation of Pichia Spp.

For biomass production, a rice-based medium is used. Approximately 200 grams of rice is mixed with 600 ml of GUY medium (glucose, urea, and yeast extract, pH 5.71) or 250 ml of concentrated GY medium (glucose and yeast extract, pH 5.69), and water. The media is spread onto stainless steel pans in a layer about 1 to 2 inches think, and sterilized.

Following sterilization, the pans are inoculated with seed culture. Optionally, added nutrients can be included to enhance microbial growth, including, for example, salts and/or carbon sources such as molasses, starches, glucose and sucrose.

Seed culture is then sprayed or pipetted onto the surface of the substrate and the trays are incubated between 25-35° C. in an enclosed reactor. Ambient air is pumped through the reactor to stabilize the temperature. Incubation for 2 to 5 days can produce 1×10⁸ to 1×10¹² cells per gram of Pichia.

The yeast and any growth by-products thereof (e.g., enzymes, solvents, and/or biosurfactants) can either be washed out and utilized in liquid form, optionally with further purification, or the yeast and substrate can be homogenized and, optionally, dried.

### Example 2—Submerged Fermentation of Pichia Spp.

The temperature and pH of the fermentation are not critical, but generally temperature should be between about 25 to less than 37° C., preferably between about 25 to 30° C. pH levels should range between about 3.0 to about 5.0, preferably between about 3.5 to about 4.5. pH stabilization during fermentation is not necessary, but it is recommended to raise pH to 3.5-4.0 if it falls below 3.0.

If necessary, control or maintenance of pH during fermentation may be accomplished using manual or automatic techniques conventional in the art, such as using automatic pH controllers for adding bases. Preferred bases employed for pH control include but are not limited to NaOH and KOH.

Both complex media such as NYDB (nutrient broth, 10 g, yeast extract, 8 g, dextrose 20 g per liter of water), PDB (potato dextrose broth), ME (malt extract) and chemically defined media supplemented with a variety of carbon source such as glucose, sucrose, sorbitol, molasses etc. can be used to produce Pichia by fermentation.

However, if necessary, the cost of the media can be further reduced by having only three major components, as listed below.

| Reagent | Concentration (g/L) | Weight (g) |
|---|---|---|
| Yeast Extract | 5 | 5,000 |
| Glucose | 30 | 30,000 |
| Urea | 1 | 1000 |

Typical growth time is 48 hours to 72 hours and CFU concentration is from 0.6 to 1.0 billion cells/ml.

The readymade culture is a final product for many applications. However, if necessary, yeast cells can be harvested by centrifugation, filtration or precipitation. The resulting yeast paste (wet biomass) can be preserved by adding salt, glycerol, lactose, trehalose, sucrose, amino acids to prolong shelf-life during storage.

### Example 3—Growth Inhibition Trial for Fusarium Oxysporum

Several treatments were examined for the ability to inhibit the growth of the Fusarium oxysporum obtained from ATCC. The treatments included Pichia anomala, Pichia anomala mutant strain (30 min under UV light and further selection), 1% SLP treatment and Pichia occidentalis.

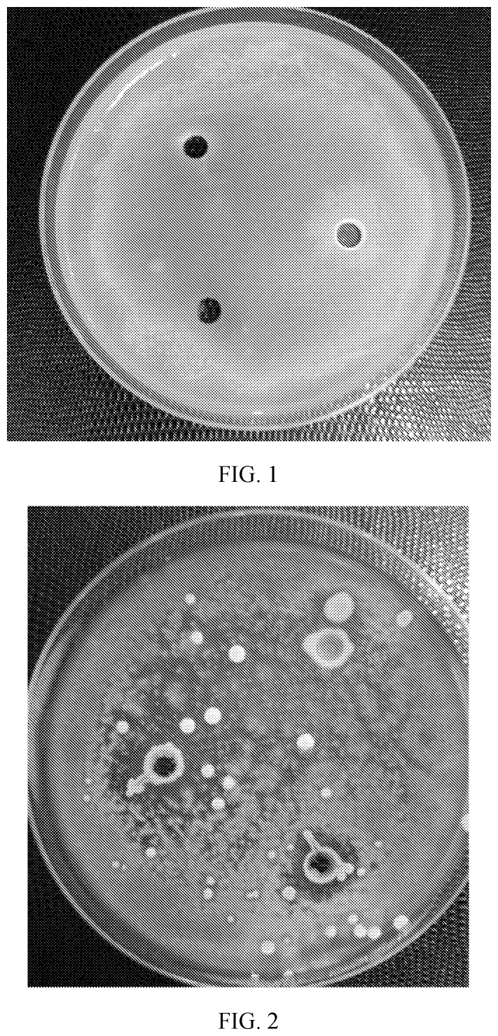

Initially, F. oxysporum were grown on agar plates and treated with a drop of treatment to observe the zones of inhibition. FIGS. 1-2 show the zones of inhibition for SLP and P. anomala, respectively.

Next, test organisms were added to liquid culture of 2-day-old F. oxysporum in flasks. Flask #1 was a control flask with only Fusarium. FIG. 3. Flask #2 contained one inoculum of P. anomala, and changes were recorded throughout the next 7 days. In flask #3, P. anomala was added every day for 3 days (10% inoculum), and the results were recorded. In flask #4, 10% SLP was added for a proof-of-principle. In flask #5, 1% SLP was added and the results were observed after 3 days.

In flask #6, P. occidentalis was added every day for 3 days (10% inoculum), and the results were recorded. In flask #7, mutant P. anomala was added every day for 3 days (10% inoculum), and the results were recorded.

Microscope slides were prepared from the culture every day after inoculation. After 3 days of treatment in the flasks, samples were also plated on agar plates to observe Fusarium growth.

### Results

All treatments, including Trichoderma, P. anomala, SLP, mutant P. anomala and P. occidentalis, had inhibiting effects on the F. oxysporum. Trichoderma competed for resources with F. oxysporum, and therefore slowed down its future growth, but did not have a direct effect on the F. oxysporum itself.

### SLP Treatment

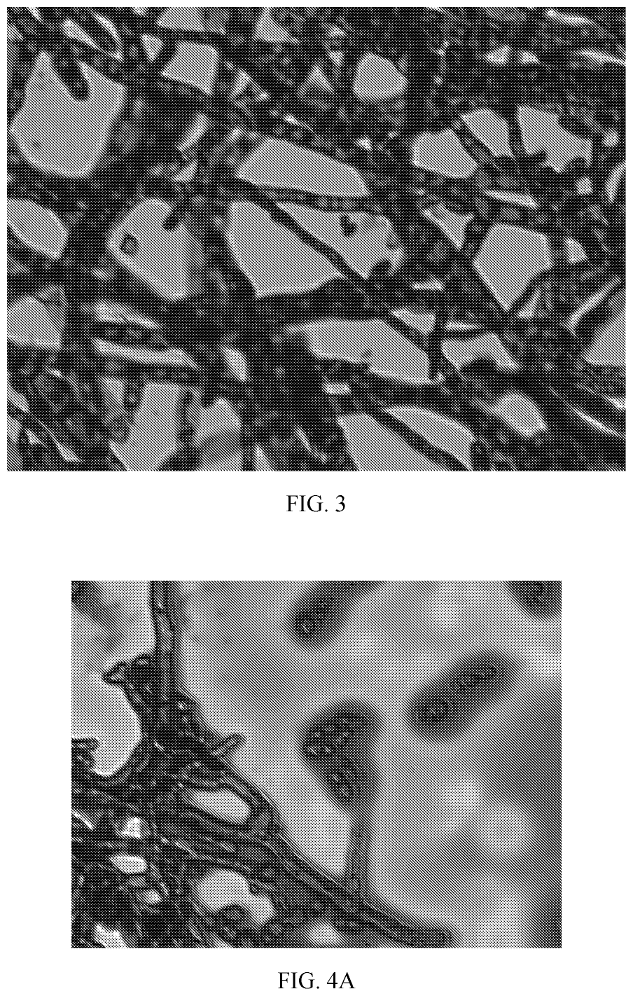

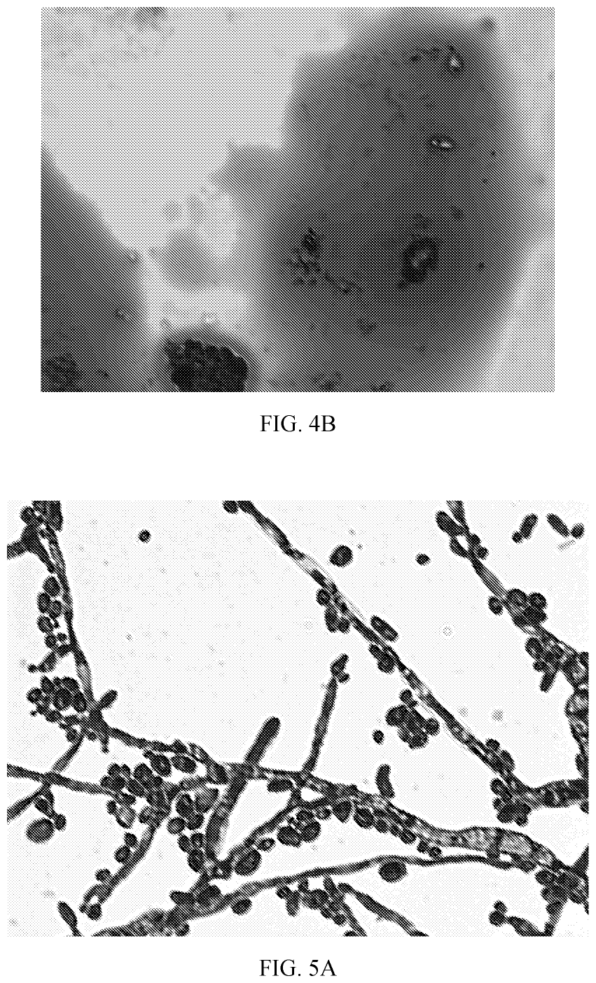

The color of the culture treated with 10% SLP turned nearly white and F. oxysporum hyphae were destroyed by the SLP. Treatment with 1% SLP three times caused destruction of the hyphae, FIGS. 4A-B, but did not have a strong effect on the spores. It worked slightly better than P. anomala.

P. anomala Treatment

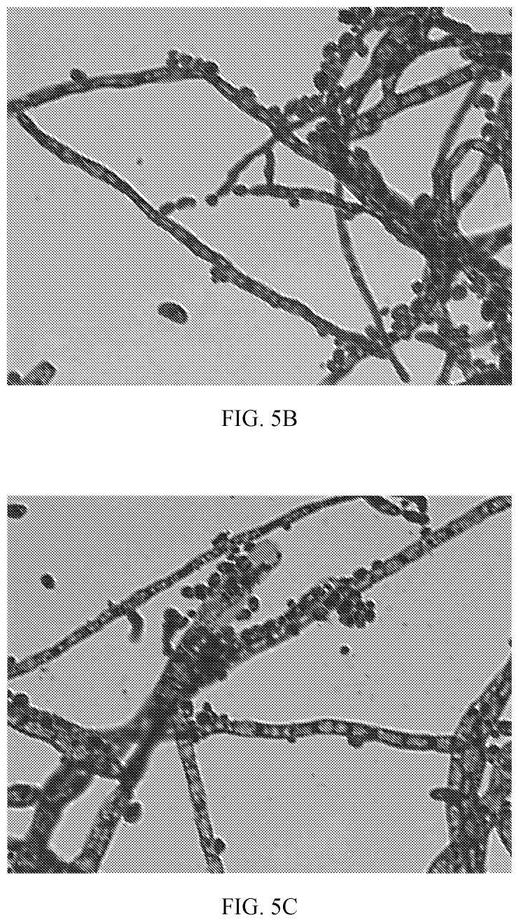

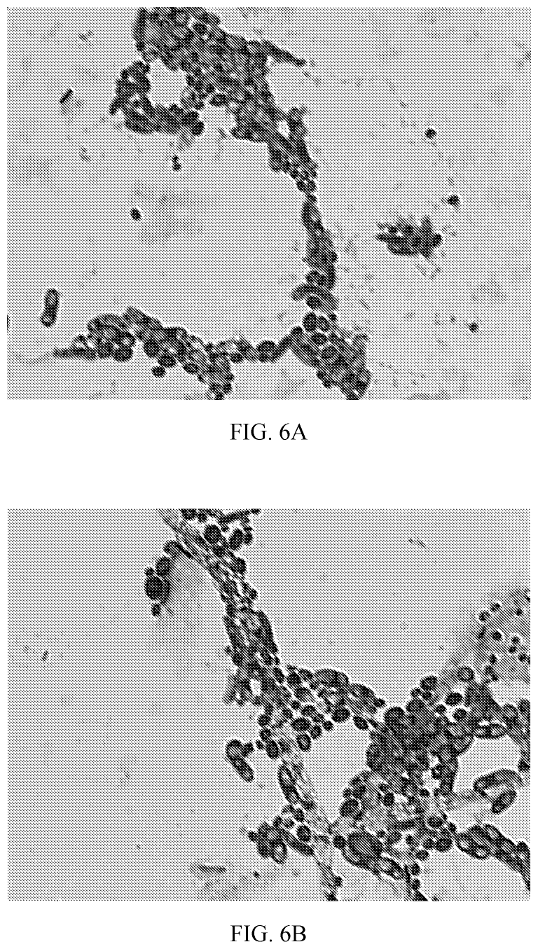

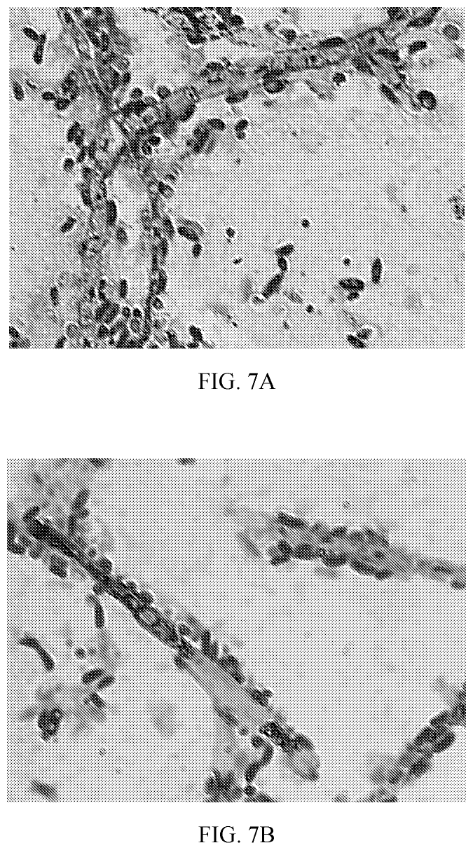

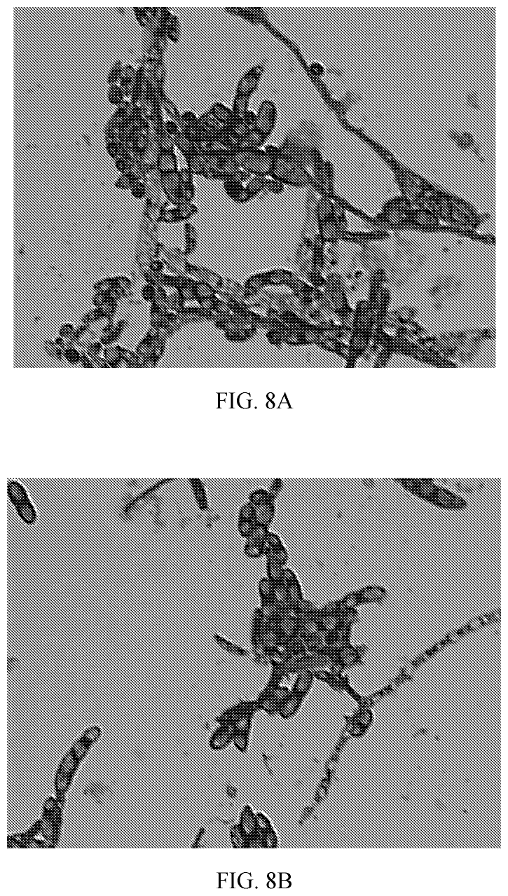

P. anomala appeared to secrete substances that destroy cell walls of F. oxysporum hyphae. it had a strong inhibiting effect on the hyphae. FIGS. 5-7. The mutant P. anomala exhibited similar effects, but somewhat faster and stronger. FIGS. 8A-B.

P. occidentalis Treatment

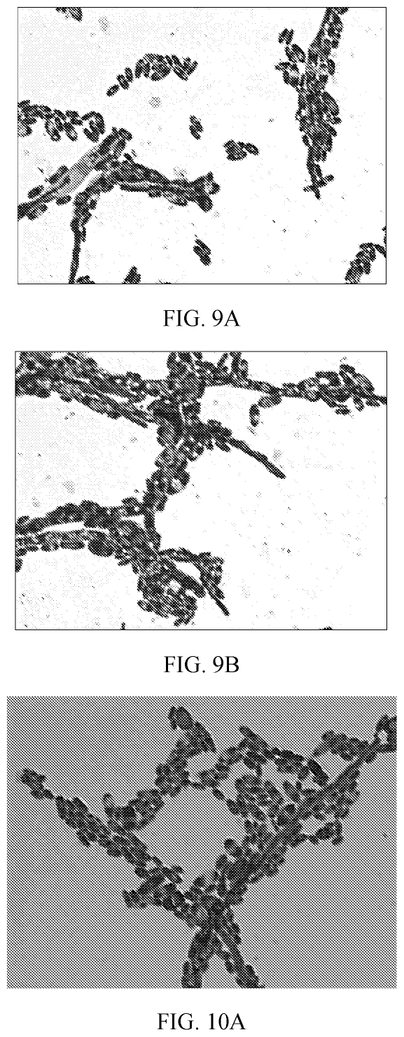

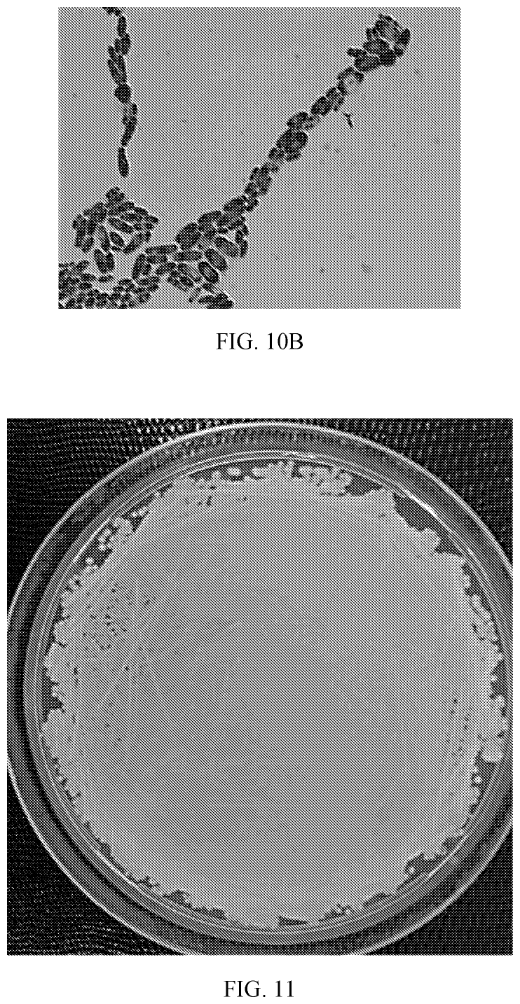

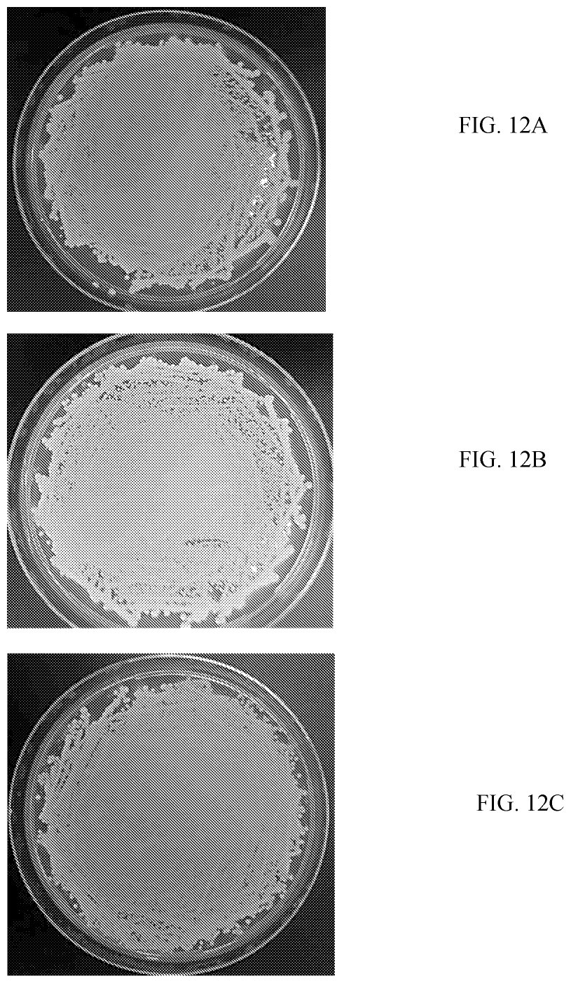

P. occidentalis exhibited the strongest killing effect on the Fusarium, both on the hyphae and on the spores, which was seen under the microscope and on the agar plates. It appeared to be secreting 2 different types of inhibiting molecules: one of which breaks the cell wall of the Fusarium and one that inhibited formation and development of new fungal hyphae from spores (FIGS. 9-12).

1. A fungicidal composition comprising one or more Pichia spp. yeasts and/or one or more growth by-products thereof, wherein the one or more Pichia spp. are selected from P. occidentalis and P. anomala, and mutants thereof.

2. The composition of claim 1, wherein the Pichia spp. yeasts are mutants of P. occidentalis and/or P. anomala, wherein the mutants are strains, genetic variants or subtypes of the Pichia spp. yeast.

3. The composition of claim 1, formulated as a soil treatment.

4. The composition of claim 3, formulated as a dry powder, dry granules, or a concentrated liquid.

5-6. (canceled)

7. The composition of claim 1, further comprising a growth medium in which the one or more Pichia spp. yeasts were produced.

8. A method of controlling and/or preventing a fungal infection in a plant, the method comprising:
applying a fungicidal composition to a plant and/or its surrounding environment, wherein the fungicidal composition comprises one or more Pichia spp. yeasts and/or one or more growth by-products thereof.

9. The method of claim 8, wherein the one or more Pichia spp. yeasts are selected from Pichia occidentalis, mutants thereof, Pichia anomala, mutants thereof, or any combination thereof.

10. (canceled)

11. The method of claim 8, wherein the step of applying the fungicidal composition comprises applying the fungicidal composition to soil in which the plant grows.

12. The method of claim 8, wherein the step of applying the fungicidal composition comprises injecting the composition into the plant's roots, trunk, stems and/or vascular system.

13. The method of claim 8, wherein the fungicidal composition is applied to the plant and/or its surrounding environment using an irrigation system.

14-16. (canceled)

17. The method of claim 8, wherein the plant is a crop plant infected by a Fusarium fungus.

18. The method of claim 17, wherein the plant is a Musa spp. plant affected by Fusarium wilt or Panama disease.

19. The method of claim 17, wherein the plant is a Coffea spp. plant.

20. The method of claim 17, wherein the plant is infected by one or more of F. avenaceum, F. bubigeum, F. culmorum, F. graminearum, F. langsethiae, F. oxysporum, F. proliferatum, F. sporotrichioides, F. poae, F. reseum, F. solani, F. tricinctum, F. verticillioides, F. virguliforme, and F. xylariodides.

21. The method of claim 8, further comprising the step of applying a biosurfactant composition to the plant and/or its environment, wherein the biosurfactant composition comprises one or more glycolipids and/or lipopeptides.

22. The method of claim 21, wherein the composition comprises a sophorolipid.

23. (canceled)

24. The method of claim 21, wherein the biosurfactant composition is injected into the plant's vascular system using a syringe.

25. (canceled)

26. A method of controlling a Fusarium infection in a plant, the method comprising: applying a biosurfactant composition to the plant and/or its surrounding environment, wherein the biosurfactant composition comprises a sophorolipid biosurfactant.

27. The method of claim 26, wherein the biosurfactant composition is injected into the plant using a syringe.

28. (canceled)

29. A method of controlling a Fusarium infection in a plant, the method comprising:
applying a fungicidal composition of claim 1 to a plant and/or its surrounding environment, and
applying a biosurfactant composition comprising a sophorolipid biosurfactant to the plant and/or its surrounding environment.

30. A method of controlling and/or preventing a Fusarium infection in an agricultural product, the method comprising:
applying a fungicidal composition of claim 1 to the agricultural product and/or its surrounding environment, and/or
applying a biosurfactant composition comprising a sophorolipid biosurfactant to the agricultural and/or its surrounding environment.

31. The method of claim 30, wherein the fungicidal composition and/or the biosurfactant composition are applied the agricultural product before harvest.

32. The method of claim 30, wherein the fungicidal composition and/or the biosurfactant composition are applied the agricultural product after harvest.

33. The method of claim 30, wherein the agricultural product is foliage, a fruit, or a vegetable.

34. (canceled)
